(19) United States
(12) Patent Application Publication
(10) Pub. No.: US 2024/0207237 A1
(43) Pub. Date: Jun. 27, 2024

(54) METHOD OF SCREENING FOR DRUG COMBINATION FOR COLORECTAL CARCINOMA TREATMENT, DRUG COMBINATION AND USE THEREOF

(71) Applicant: National Tsing Hua University, Hsinchu City (TW)

(72) Inventors: Fan-Gang Tseng, Hsinchu City (TW); Hsin-Yu Yang, Hsinchu City (TW); Yi-Chi Hung, Hsinchu City (TW); Ya-Wen Cheng, Hsinchu City (TW); Masturah Mohamed Abdul Rashid, Hsinchu City (TW); Venkanagouda S. Goudar, Hsinchu City (TW)

(73) Assignee: National Tsing Hua University, Hsinchu City (TW)

(21) Appl. No.: 18/329,586

(22) Filed: Jun. 6, 2023

(30) Foreign Application Priority Data
Dec. 22, 2022 (TW) 111149353

(51) Int. Cl. A61K 31/4412; A61K 31/7068; A61K 31/7115; A61P 35/00

(52) U.S. Cl. CPC A61K 31/4412; A61K 31/7068; A61K 31/7115; A61P 35/00

(57) ABSTRACT

A method of screening for a drug combination for treating colorectal carcinoma, the screened drug combination and its related application are provided. The drug combination includes at least three selected from Regorafenib, Gemcitabine, Cetuximab and 5-Fluorouracil. With the method of the disclosure, the best and novel drug combination can be selected from the existing clinical drugs, the technical effect of rapidly screening for a drug combination can be achieved, and the screened combination drug has an excellent therapeutic effect.

### CROSS-REFERENCE TO RELATED APPLICATION

This application claims the priority benefit of Taiwan application serial no. 111149353, filed on Dec. 22, 2022. The entirety of the above-mentioned patent application is hereby incorporated by reference herein and made a part of this specification.

### BACKGROUND

### Technical Field

The present disclosure relates to a method of screening for a drug combination, and particularly relates to a method of screening for a drug combination for treating colorectal carcinoma, the screened drug combination and its related medical application.

### Description of Related Art

In recent years, colorectal carcinoma (CRC) is one of the three most common cancers in the world, and the lifetime risk of developing CRC is about 13.9%. The existing treatment method for colorectal carcinoma mainly includes chemotherapy (i.e., administration of chemotherapy drugs) and targeted therapy (i.e., administration of targeted drugs). However, since the single-drug therapy for cancer is prone to drug resistance, the efficacy of the existing chemotherapy drugs has reached a plateau, leading to difficulty in progression. Therefore, in addition to developing new drugs, combining the existing drugs to achieve effective therapeutic effects is one of the important therapeutic strategies. However, drug combination screening is not easy and often consumes a lot of manpower and time. Therefore, there is a need for a method of screening for a drug combination quickly and effectively.

### SUMMARY

The disclosure relates to a method of screening for a drug combination (or called “drug combination screening method” in some examples). The method of the disclosure can screen out a specific and effective drug combination (or called “combinational drug” in some examples) from the existing clinical drugs, and the screened drug combination provides an excellent therapeutic effect. The screened drug combination of the present disclosure can be prepared as a drug for treating colorectal carcinoma, and its application includes the medical use of preparing a drug for treating human colorectal carcinoma.

The disclosure provides the application of a drug combination in the preparation of a drug for treating human colorectal carcinoma, wherein the drug combination includes at least three selected from Regorafenib, Gemcitabine, Cetuximab and 5-Fluorouracil.

In an embodiment of the present disclosure, the drug combination has a cytostatic effect of inhibiting survival of 55% of cancer cells.

In an embodiment of the present disclosure, when the drug combination includes regorafenib, gemcitabine and 5-fluorouracil, the drug combination has a cytostatic effect of inhibiting survival of 70% of cancer cells.

In an embodiment of the present disclosure, when the drug combination includes regorafenib, gemcitabine, cetuximab and 5-fluorouracil, the drug combination has a cytostatic effect of inhibiting survival of 80% of circulating tumor cells.

The disclosure provides a method of screening for a drug combination, wherein the drug combination is suitable for treating a cancer, and the method includes: providing cell lines and clinical drugs corresponding to a cancer; applying the clinical drugs to the cell lines respectively, so as to measure a IC50 value and an initial cell viability of the corresponding clinical drug in each of the cell lines; using an artificial neural network to take the IC50 values and the initial cell viability values as a data set, fitting a regression function to generate a parabolic surface, providing a quantitative relationship between drug dosage and efficacy, and obtaining output cell viability values of the clinical drugs; selecting and combining the clinical drugs to generate a plurality of drug combinations, using an artificial neural network, fitting a regression function to generate a parabolic surface, obtaining output cell viability values of the drug combinations, and generating a rank table; screening for a specific drug combination using the rank table; and verifying the specific drug combination with a mouse model and the cell line corresponding to the cancer.

The present disclosure provides a drug combination including at least three selected from regorafenib, gemcitabine, cetuximab and 5-fluorouracil.

Based on the above, in the present disclosure, with the aid of an Artificial Intelligence-Phenotypic Response Surface (AI-PRS) platform for in vitro (non-living) drug screening, the best and novel drug combination can be selected from the existing clinical drugs, the technical effect of rapidly screening for a drug combination can be achieved, and the screened combination drug has an excellent therapeutic effect.

In order to make the above-mentioned features and advantages of the present disclosure more comprehensible, the following specific embodiments are described in detail together with the accompanying drawings.

### BRIEF DESCRIPTION OF THE DRAWINGS

FIG. 1 is a schematic flow chart of a method of screening for a drug combination according to an embodiment of the present disclosure.

FIG. 2 is a phenotypic response surface simulated by using an AI-PRS platform to predict the best two drug combinations in different cell lines according to an embodiment of the present disclosure.

FIG. 3 is the result of cell viability values of a patient-derived xenograft (PDX) model treated with drug combinations according to an embodiment of the present disclosure.

FIG. 4 is the result of cell viability values of CTCs treated with drug combinations according to an embodiment of the present disclosure.

### DESCRIPTION OF THE EMBODIMENTS

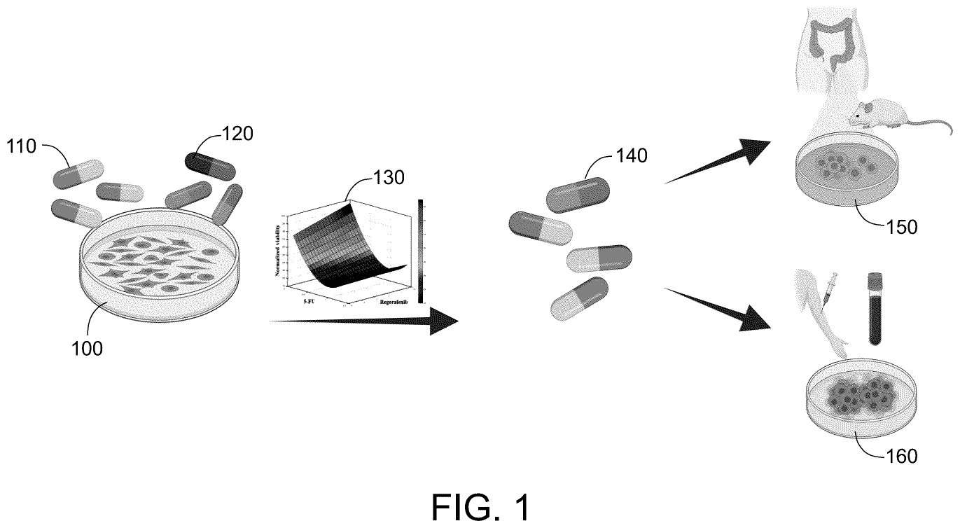

FIG. 1 is a schematic flow chart of a method of screening for a drug combination according to an embodiment of the present disclosure.

Referring to FIG. 1, cell lines 100 and clinical drugs 110 and 120 are provided. Next, clinical drugs 110 and 120 are respectively applied to the provided cell lines 100 to measure the IC50 value and the cell viability (or called “cell survival rate” in some examples) of each of the clinical drug 110 or 120 in each cell line 100. Specifically, this embodiment includes selecting the existing clinical drugs and applying them to the provided cell lines, the clinical drugs may include chemotherapy drugs (or called “chemotherapeutic agents” in some examples) 110 and/or target drugs (or called “target therapeutic agents” in some examples) 120.

In this example, the cell lines 100 include four human colorectal carcinoma cell lines purchased from the American Type Culture Collection (ATCC); namely HCT116, HT29, LoVo and SNUC1, but the disclosure is not limited thereto. The clinical drugs 110 include five chemotherapy drugs commonly used in the treatment of human colorectal carcinoma. The clinical drugs 110 include 5-Fluorouracil (5-FU), Oxaliplatin, Gemcitabine, Irinotecan and Folinic acid. The clinical drugs 120 include five target drugs commonly used in the treatment of human colorectal carcinoma. The clinical drugs 120 include Regorafenib, Lenvatinib, Bevacizumab, Cetuximab, and Panitumumab, but the disclosure is not limited thereto. Table 1 shows the clinical drugs used in this example and their representative symbols.

TABLE 1
Clinical drugs and representative symbols

| Clinical Drugs | Representative Symbols |
|---|---|
| 5-Fluorouracil (5-FU) | U |
| Oxaliplatin | O |
| Gemcitabine | G |
| Irinotecan | I |
| Folinic acid | F |
| Regorafenib | R |
| Lenvatinib | L |
| Bevacizumab | B |
| Cetuximab | C |
| Panitumumab | P |

In this embodiment, the experimental method of this step can be summarized as follows:

### [Cell Culture]

Cells were cultured in McCoy's 5A medium for HCT116 and HT29, F-12K medium for LoVo, RPMI medium for SNUC1, containing 10% heat-inactivated fetal bovine serum and 100 units (U)/mL penicillin, in a humidified atmosphere of 5% CO₂ at 37° C. All the mediums were purchased from Dow Corning (Corning life sciences, Dow Corning, USA).

### [IC50 and Cell Viability Assay of Clinical Drugs]

To determine suitable cell seeding number for each cell line, different number (300, 500, 1000, 3000) of cells/wells were seeded in a 96-well plate. A cell viability was determined using a real-time viability assay (RealTime-Glo™ Assay Reagent). Luminescence intensities were measured at different time points (4, 8, 16, 24, 36, 48, 60 and 72 hours) to evaluate a cell growth curve. To establish IC50 values of ten drugs on each cell line, gradient concentrations of each drug were prepared. 5-FU, Oxaliplatin, Gemcitabine, Irinotecan, Leucovorin, Regorafenib, Lenvatinib are small molecule drugs, which were prepared with final concentrations of 100, 50, 25, 10, 5, 1, 0.1, 0.01 and 0.001 μM; and Bevacizumab, Cetuximab, Panitumumab are macromolecular drugs, which were prepared with the final concentrations of 1, 0.5, 0.25, 0.1, 0.05, 0.01, 0.001, 0.0001 and 0.00001 μM. The cell viability was measured with Cell Counting Kit 8 (e.g., CCK-8 purchased from Dojindo molecular technologies, Japan), and cell viability assay was performed after 2 hours of incubation by measuring the absorbances at 450 nm. Relative cell numbers were determined as compared with the control cells. All experiments were performed in triplicate manner, and data were represented as expressed as mean±standard deviation (mean±SD). Table 2 lists the IC50 values of clinical drugs on HCT116 cell lines, wherein the term “Resistance” means that the clinical drug does not affect the cell viability of HCT116, indicating that HCT116 is resistant to the specific drug.

TABLE 2
IC50 values of clinical drugs on HCT116 cell lines

| Clinical Drugs | IC50 (μM) ± SD |
|---|---|
| 5-FU (U) | 8.561 ± 0.897 |
| Oxaliplatin (O) | 10.28 ± 3.273 |
| Gemcitabine (G) | 0.1832 ± 1.32 |
| Irinotecan (I) | 0.01 ± 0.03 |
| Folinic acid (F) | Resistance |
| Regorafenib (R) | 5.888 ± 0.713 |
| Lenvatinib (L) | 28.47 ± 0.802 |
| Bevacizumab (B) | Resistance |
| Cetuximab (C) | Resistance |
| Panitumumab (P) | Resistance |

Continue referring to FIG. 1, an appropriate drug combination 140 is screened out using an Artificial Intelligence-Phenotypic Response Surface (AI-PRS) platform 130 for calculation. Specifically, in this embodiment, the IC50 and the cell viability of each of the clinical drugs 110 and 120 in the cell lines 100 are input into the AI-PRS platform, and through the intelligent computing system of the platform, the AI-PRS platform 130 links drug and dose/dosage inputs to phenotypic outputs through a quadratic algebraic equation, so as to dynamically and individually evaluate and optimize combinational drug therapies.

The AI-PRS platform is based on combining different selection principles and optimizing different selection principles to quantify the interaction and efficacy of various drugs, so as to develop a better combination therapy to treat the current or future disease symptoms. Based on the understanding of interaction and interacting between drugs, phenotypic response surface fits a parabolic surface to a set of drug dosages and biomarkers, and quadratic surface modeling can be used to understand the effect of the set of drugs to the measured biomarkers. The results (curve surfaces) generated by this platform allow researchers to quickly bypass the need for in vitro screening of multiple drug combinations that respond to a patient's unique phenotype. For the need to develop a specific therapy under time constraints, or even a personalized therapy for a special individual, the drug combination screening method of the disclosure can use small data sets to establish a novel drug treatment mechanism.

Specifically, the drug combinatorial arrays were adapted from the orthogonal array composite design (as previously described by Xu et al.), including the minimum number of combinations required to fully interrogate the intended search space. For in vitro (non-living) experiments, 155 combinations were experimentally tested within a 10-drug search space, at three different concentrations (IC₀, IC₁₀ and IC₂₀) respectively inhibiting the cell viability values of 0%, 90% and 80% after the cells were given drugs. For in vitro experiments, the relative viability of the cells were used as experimental data points for fitting.

AI-PRS uses an artificial neural network to take IC50 values and relative viability values of the cells (i.e., initial cell viability values) as a data set, and fits a regression function to generate a parabolic surface, and therefore provides a quantitative relationship between drug dosage and efficacy. Herein, the efficacy of the drug or drug combination is expressed by calculating the output cell viability (e.g., “Output (% Viability)” in Table). In the method of using an artificial neural network with AI-IPS, clinical drugs that may be used as a single drug or combined with each other as a drug combination, with the aforementioned method, the IC50 value and the relative viability values of the cells (i.e., the initial cell viability values) are used as a data set, and the regression function is fitted to generate a parabolic surface to obtain the output cell viability of a single drug or a combination of multiple drugs. Based on the obtained efficacy values, a rank table (or called “ranked list” in some examples) is generated and ranked according to the efficacy of each drug combination, wherein the higher the rank (ranking) in the rank table, the better the efficacy of the drug or drug combination. Afterwards, according to established principles, the specific drug combination is screened out using the rank table. Moreover, the specific drug combination can be further experimented with mouse PDX model and circulating tumor cells to verify the efficacy of the drug combination.

Based on the above, AI-PRS generates rank tables (e.g., Table 3 and Table 4) of all the possible permutations of drug combinations, from top to worse ranked. With the cell viability values from portions of the experimented drug combinations, the predictive power of the generated outcome is calculated as correlation coefficients, ranging in value between 0 and 1, to confirm the fidelity of AIPRS optimization (derived from the experimental output values and projected output values for the corresponding drug combinations), wherein a number close to 1 indicates that the prediction is completely certain and accurate, while a number of 0 indicates that the prediction is not certain and accurate. The clinical drugs listed have their respective concentrations, wherein the IC10 of Cetuximab is represented by C1, the IC20 is represented by C2, and so on.

In this example, the results of this step are outlined below.

The AI-PRS based 155 drug-dose combinations were tested on cell lines, the top ten high efficacy drug-dose combinations (1, 2, 3, and 4 drug-dose combinations) were selected, and the results were recorded in Table 3. Further, the results were compared with standard of care (SOC) treatments (Rank 131, 242, 997, 1348 in Table 3). In Table 3, the multi-drug combinations (combining 2 drugs, 3 drugs or 4 drugs) screened out in this example have higher curative effects than single drug treatment and SOC drug treatment. Among them, the survival rate of the combination of four drugs is the lowest. In Table 3, the effect of standard of care on HCT116 (non-metastatic cell line) is not as high as expected, and even ranks behind the thousand. The above results also appeared in other cell lines (not shown), indicating that the effect of SOC on cell lines is not good. In addition, from the output results of single drugs in Table 3, the top three drugs with the high curative effects on HCT116 cell lines are 5-FU, Regorafenib, and Gemcitabine.

TABLE 3
AI-PRS outputs on HCT116 cell lines

| | | Output | | | Output |
|---|---|---|---|---|---|
| | | (% | | | (% |
| Rank | Drug | Viability) | Rank | Drug | Viability) |
| 1 | U2 | 19.57 | 1 | U2G2 | 2.25 |
| 2 | R2 | 29.00 | 2 | U2R2 | 6.13 |
| 3 | U1 | 33.53 | 3 | U2L2 | 7.67 |
| 4 | G2 | 42.38 | 4 | U1R2 | 7.80 |
| 5 | R1 | 43.00 | 5 | U1G2 | 9.34 |
| 6 | G1 | 67.02 | 6 | U2R1 | 9.72 |
| 7 | L2 | 69.60 | 7 | U2O1 | 12.45 |
| 8 | O2 | 70.52 | 8 | U2G1 | 13.15 |
| 9 | O1 | 71.92 | 9 | G2R2 | 13.23 |
| 10 | L1 | 75.65 | 10 | U2C1 | 13.72 |
| 1 | U2R2L2 | −5.76 | 1 | U2R2L2C2 | −11.60 |
| 2 | U2O1G2 | −4.87 | 2 | U2O1G2C1 | −10.71 |
| 3 | U2G2L2 | −4.43 | 3 | U2G2L2C1 | −10.27 |
| 4 | U1R2L2 | −4.10 | 4 | U2O1G2L2 | −10.09 |
| 5 | U2G2C1 | −3.60 | 5 | U2O1R2L2 | −10.04 |
| 6 | U2G2P2 | −2.60 | 6 | U1R2L2C1 | −9.94 |
| 7 | U2R1L2 | −2.18 | 7 | U2O1G2P2 | −9.71 |
| 8 | U2G2L1 | −0.58 | 8 | U1O1R2L2 | −9.15 |
| 9 | U2O2G2 | −0.57 | 9 | U2G2C1P2 | −8.44 |
| 10 | U2R2C2 | 0.29 | 10 | U2R1L2C1 | −8.02 |
| 131 | FOLFOX | 13.32 | 997 | FOLOFIRI + Cetux | 15.29 |
| 242 | FOLFIRI | 21.13 | 1348 | FOLOFIRI + Beva | 21.13 |

Besides, LoVo is a metastatic CRC cell line. The top 10 drug combinations with the best efficacy for single drug, 2-drug combination, 3-drug combination, and 4-drug combination are shown in Table 4. The rankings of target drugs (Cetuximab and Panitumumab) move forward both in the outputs of single drugs and combination drugs. Moreover, the efficacy of the SOC treatment with target drugs is better than the efficacy of the SOC treatment with chemotherapy drugs (FILFIRI and FILFOX) only, indicating improved efficacy of targeted drugs against metastatic cancer cells. In addition, the top three drugs with efficacy on LoVo cell lines are 5-FU, Cetuximab and Lenvatinib. However, Cetuximab is considered to be drug-resistant in IC50 determination (as shown in Table 2), indicating that the drugs have synergistic or antagonistic effects with each other in combination drugs.

TABLE 4
AI-PRS outputs on LoVo cell lines

| | | Output | | | Output |
|---|---|---|---|---|---|
| | | (% | | | (% |
| Rank | Drug | Viability) | Rank | Drug | Viability) |
| 1 | U2 | 19.05 | 1 | U2L1 | 10.62 |
| 2 | U1 | 45.41 | 2 | U2C1 | 12.26 |
| 3 | C2 | 78.72 | 3 | U2P2 | 15.04 |
| 4 | C1 | 91.49 | 4 | U2C2 | 15.06 |
| 5 | L2 | 92.40 | 5 | U2L2 | 15.16 |
| 6 | R2 | 94.43 | 6 | U212 | 15.88 |
| 7 | L1 | 98.89 | 7 | U2R2 | 17.12 |
| 8 | R1 | 103.41 | 8 | U2R1 | 18.01 |
| 9 | 12 | 105.37 | 9 | U2I1 | 18.80 |
| 10 | P2 | 109.83 | 10 | U2B1 | 19.05 |
| 1 | U2L1C1 | 3.83 | 1 | U1R2L1C2 | −1.41 |
| 2 | U2L1P2 | 6.61 | 2 | U1R2L2C2 | −0.14 |
| 3 | U2L1C2 | 6.63 | 3 | U1I2L2C2 | 0.45 |
| 4 | U1L2C2 | 6.96 | 4 | U2I2L1C1 | 0.66 |
| 5 | U2I2L1 | 7.45 | 5 | U2L1C1P2 | 2.24 |
| 6 | U2L2C1 | 8.37 | 6 | U1I2L1C2 | 2.84 |
| 7 | U1R2C2 | 8.96 | 7 | U1R1L2C2 | 3.14 |
| 8 | U2I2C1 | 9.09 | 8 | U2I2L1P2 | 3.44 |
| 9 | U1L1C2 | 9.35 | 9 | U2I2L1C2 | 3.46 |
| 10 | U2R2C1 | 10.33 | 10 | U1R1L1C2 | 3.57 |
| 77 | FOLFIRI | 16.63 | 99 | FOLFIRI + Cetux | 9.84 |
| 117 | FOLFOX | 19.80 | 444 | FOLFIRI + Beva | 16.63 |

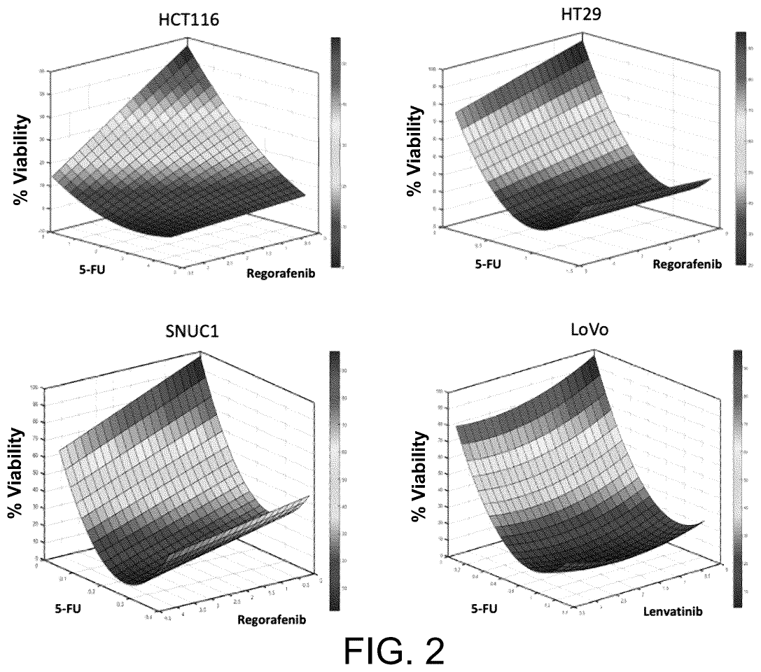

FIG. 2 is a phenotypic response surface simulated by using an AI-PRS platform to predict the best two drug combinations in different cell lines according to an embodiment of the present disclosure.

In this example, although different cell lines (HCT116, HT29, SUNC1, LoVo) are all derived from CRC patients, there are differences in the type of disease, gender, metastasis status, to the molecular features and gene sequences within the cells. However, heterogeneity is the biggest challenge in the cancer treatment. Hence, conventional combinatorial therapies fail to treat cancer efficiently. The AI-PRS of this embodiment implicitly represents the integration of disease mechanisms and drug activities for a particular biological system. In addition, the phenotypic response surface can be simulated through the PRS coefficient by the output results. As shown in FIG. 2, the PRS drug activity surface based on the prospective treatment of two best combination drugs are plotted for 4 CRC cell lines, wherein the horizontal axis represents the drug concentration and the vertical axis represents the cell viability (i.e., “% Viability”). The efficacies of the drug combinations are very different, especially on the metastatic cell lines with much higher viability values. The very smooth surface indicates the synergistic activity of both the drugs and doses against 4 cell lines. This characteristics confirms the hypothesis of quadratic algebraic equation on the drug response to bio-systems. However, the activity and phenotypic response surface of both the drugs are different on 4 cell lines, which illustrates the different mechanisms between cell lines and drugs. Besides, the real-time drug response to the tested biological system gives different phenotypic surfaces at different time points, and they change dynamically during the treatment, allowing continuous optimization during the time of treatment.

In this step, the drugs with the best efficacy (including 4 chemotherapeutic drugs (5-FU, Gemcitabine, Irinotecan, Oxaliplatin) and 2 target drugs (Regorafenib, Cetuximab)) common to the 4 cell lines are screened for further verification to determine the best combination of drugs.

Continue referring to FIG. 1, the screened drug combination is tested with a mouse model 150 (e.g., mouse PDX model) and patients' circulating tumor cells (CTCs) 160 to confirm the best combination of drugs. Since the dosage effects of preclinical drugs in the screening test are often inconsistent with the results of clinical trials, in this example, it is necessary to further verify the efficacy of actual clinical drugs through the mouse PDX model and circulating tumor cells (CTCs) of patients.

The PDX model represents a patient-derived tumor xenograft (PDX) model, which is a transplanted tumor model formed by implanting tumor tissues and primary cells derived from a patient into immune-deficient mice. The PDX model has become an attractive platform in translational cancer medicine, which can recapitulate the original patients tumor characteristics more accurately as compared to the conventional cell lines. The PDX model is like an in vivo tumor model, and provides unique advantages in preclinical drug screening and precision medicine. Advantageously, the accuracy of the PDX model and corresponding patient response to treatment has been demonstrated in several studies.

On the other hand, circulating tumor cells (CTCs) are cancer cells that detach from the primary tumor and enter blood vessels to wait for opportunities to metastasize to various parts of the body with the blood circulation, and thus, CTCs are closely related to cancer metastasis. Since the expansion of CTCs is directly obtained and derived from patients, it has a very high correlation with the actual physiological status of the patient in situ. Therefore, the expansion of CTCs is an important evidence of disease progression in an in vitro drug testing platform that simulates patients. The expansion of CTCs also provides clear evidence for drug resistance mechanisms. Therefore, CTCs are not only used as real-time cancer markers to monitor cancer progression, disease recurrence, and clinical staging, but also used in combination drug trials to understand the efficacy of drugs. In addition, combination drug trials of CTCs play a critical role in assessing the efficacy and safety of diagnostics and will also leverage population-wide data to identify personalized treatment strategies.

In this step, the experimental methods and results of validating drug combinations using PDX models and CTCs are summarized as follows:

[Validation of Drug Combinations with PDX Model]

Tumor tissues were collected from CRC patients (stage IIIB) with a genetic background such as BRAF gene mutation and implanted into NOD-SCID mice. The first generation of mice that received patient tumor fragments is usually denoted as F0. When the tumor burden in F0 mice became too great, the tumors were passed on to the next generation of mice. The subsequent generations of mice are denoted as F1, F2, F3 . . . Fn. Tumor tissues were collected to establish the primary cultured cells for each generation of mice. These cells are used to identify biomarkers and screen drugs. For anticancer drug screening, cells after F3 generation are used, as these cells are usually used after ensuring that the PDX does not diverge genetically or histologically from the patient's tumor. F4 PDX cells were cultured in 96-well culture plates in a humidified atmosphere of 5% CO₂ at 37° C. with a primary cell culture solution. PDX was treated with AI-PRS based 6 drug-dose combinations and different doses (IC10, IC30) of 5-FU, Gemcitabine, Regorafenib, Cetuximab, Oxaliplatin, and Irinotecan. Cells were incubated with drug-dose combinations for 72 hours, followed by measurement of cell viability using a Fluorescence-based viability assay (LIVE/DEAD® Viability/Cytotoxicity Kit, Thermo Fisher). A relative survival rate was calculated as the percentage of drug-treated cells as compared to untreated cells.

[Validation of Drug Combinations with CTCs]

Peripheral venous blood (30 mL) were collected from stage IVA colon cancer patients in EDTA anticoagulated tubes, added to centrifuge tubes with Ficoll-Paque Plus and centrifuged to obtain a CTC-containing PBMC fraction. The pellet was resuspended in phosphate-buffered saline (PBS) containing 1% BSA, 2 mM EDTA and CTCs were enriched by RosetteSep™ CTC Enrichment Cocktail kit (Stem cell technologies, Cambridge, MA, USA). The enriched cells were obtained and suspended in DMEM/F12 medium containing EGF, bFGF, B27 supplements and platelet lysate.

CTCs spheroids were cultured for 20 days and then transferred to 96-well culture plates without disturbing them for drug susceptibility assay. The CTCs were treated with AI-PRS based 10 drug-dose combinations and different doses (IC15, IC20, IC30) of 5-FU, Gemcitabine, Regorafenib, Cetuximab were used. Cells were incubated with drug-dose combinations for 72 hours, and cell viability was measured using a Fluorescence-based viability assay (LIVE/DEAD® viability/cytotoxicity kit, Thermo Fisher). A relative survival rate (or called “relative cell viability” in some examples) was calculated as the percentage of drug-treated cells as compared to untreated cells.

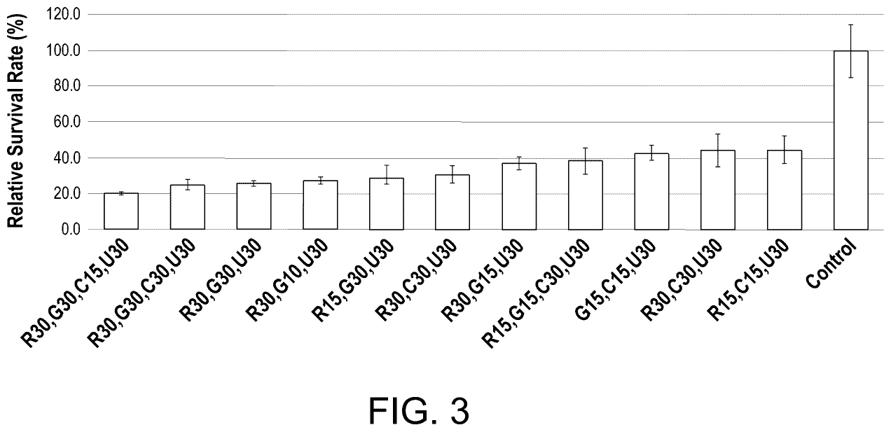

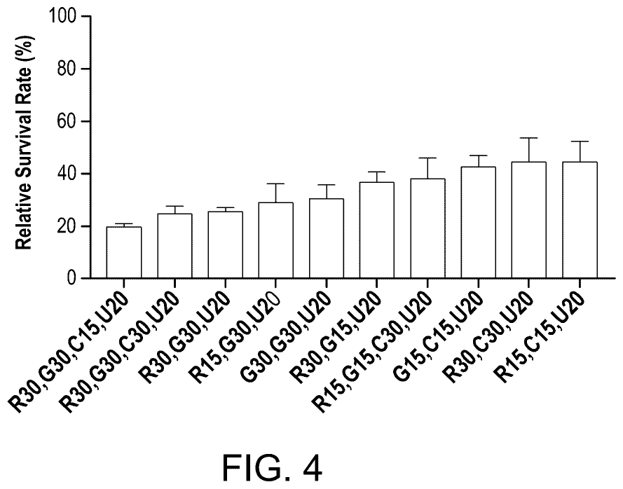

FIG. 3 is the result of cell viability values of a PDX model treated with drug combinations according to an embodiment of the present disclosure. FIG. 4 is the result of cell viability values of CTCs treated with drug combinations according to an embodiment of the present disclosure.

AI-PRS based drug-dose combinations were tested on the PDX model and CTCs, and the results are shown in FIG. 3 and FIG. 4, respectively. The drug combination of 5-Fluorouracil (U), Gemcitabine (G) and Regorafenib (R) is one of the effective combinations, no matter in the PXD model or CTC test, such drug combination can inhibit about 73% relative survival rate/relative cell viability for CTC and PDX model cell lines. In addition, as shown in FIG. 4, the drug-dose combination of R30, G30, C15, and U20 becomes a more effective drug combination, which can inhibit about 80% relative survival rate/relative cell viability. Furthermore, the relative survival rates of the remaining drug-dose combinations are approximately lower than 45%.

Drug activity depends on numerous pathways in the network, and thus, drug dosage parameters cannot be effectively designed using conventional methods. Traditional combination therapies ultimately lead to poor treatment outcomes, toxicity and side effects. Therefore, there is still a need to study effective drug-dose combinations to treat dynamic diseases such as cancer. Cumulative drug-dose and nonoverlapping drug mechanism of action are important factors in minimizing treatment effects. In the present disclosure, the AI-PRS platform is successfully applied to conduct in vitro experiments (colorectal carcinoma cell lines, PDX models and CTCs) to find the optimal drug-dose combination. The AI-PRS platform optimizes effective drug-dose combinations without reference to molecular biological response pathways and drug interaction data. With the aid of the AI-PRS platform, effective 1, 2, 3, and 4 drug-dose combinations are screened out from in vitro simulation experiments. Among hundreds of combinations, Regorafenib(R)/Gemcitabine(G)/Cetuximab(C)/5-Fluorouracil(U) combinations show excellent activities for all in vitro experiments. The combination of 3 drugs (R/G/U) shows the highest toxicity to PDX model cell lines. In addition, the combination of 4 drugs (R/G/C/U) shows the highest toxicity (i.e., the lowest cell viability) on circulating tumor cells (CTCs) cultured in vitro. The present disclosure demonstrates the prospective verification of the AI-PRS platform, provides an accurate and efficient drug combination screening method, helps to determine effective drug combinations and/or dosage ratios for the treatment of colorectal and colorectal carcinoma, and optimizes and determines the potential usage of drug combinations, so as to achieve high curative effect in medical application.

In summary, in the present disclosure, with the aid of an AI-PRS platform for in vitro (non-living) drug screening, the best and novel drug combination is screened out from the existing clinical drugs, without additional reference to data such as molecular biological reaction pathways and drug interactions. The technical effect of rapidly screening for a drug combination can be achieved, and the screened combination drug has an excellent therapeutic effect.

Although the present disclosure has been disclosed above with the embodiments, it is not intended to limit the present disclosure. Anyone with ordinary knowledge in the technical field may make some changes and modifications without departing from the spirit and scope of the present disclosure. The scope of protection of the present disclosure should be defined by the scope of the appended patent application.

What is claimed is:

1. A use of a drug combination in preparation of a drug for treating a human colorectal carcinoma, wherein the drug combination comprises at least three selected from regorafenib, gemcitabine, cetuximab and 5-fluorouracil.

2. The use according to claim 1, wherein the drug combination has a cytostatic effect of inhibiting survival of 55% of cancer cells.

3. The use according to claim 1, wherein the drug combination comprises the regorafenib, the gemcitabine and the 5-fluorouracil, and the drug combination has a cytostatic effect of inhibiting survival of 70% of cancer cells.

4. The use according to claim 1, wherein the drug combination comprises the regorafenib, the gemcitabine, the cetuximab and the 5-fluorouracil, and the drug combination has a cytostatic effect of inhibiting survival of 80% of circulating tumor cells.

5. A method of screening for a drug combination, the drug combination is suitable for treating a cancer, the method comprising:
providing cell lines and clinical drugs corresponding to a cancer;
applying the clinical drugs to the cell lines respectively, so as to measure a IC50 value and an initial cell viability of the corresponding clinical drug in each of the cell lines;
using an artificial neural network to use the IC50 values and the initial cell viability values as a data set, fitting a regression function to generate a parabolic surface, providing a quantitative relationship between drug dosage and efficacy, and obtaining output cell viability values of the clinical drugs;
selecting and combining the clinical drugs to generate a plurality of drug combinations, using an artificial neural network, fitting a regression function to generate a parabolic surface, obtaining output cell viability values of the drug combinations, and generating a rank table;
screening for a specific drug combination using the rank table; and
verifying the specific drug combination with a mouse model and the cell line corresponding to the cancer.

6. The method according to claim 5, wherein the cancer is a human colorectal carcinoma, and the specific drug combination is selected from at least three of regorafenib, gemcitabine, cetuximab and 5-fluorouracil.

7. A drug combination comprising at least three selected from regorafenib, gemcitabine, cetuximab and 5-fluorouracil, wherein the drug combination is suitable for treating a cancer.

8. The drug combination according to claim 7, wherein the cancer is a human colorectal carcinoma.
